(19) United States
(12) Patent Application Publication
(10) Pub. No.: US 2002/0025548 A1
(43) Pub. Date: Feb. 28, 2002

(54) PROCESS FOR THE RECOVERY OF CRYSTALLINE BETA-CAROTENE FROM A NATURAL SOURCE

(76) Inventors: MIEKE SIBEYN, AMERSFOORT (NL); ROBERTUS MATTHEUS DE PATER, DELFT (NL)

Correspondence Address: MORRISON & FOERSTER LLP, 3811 VALLEY CENTRE DRIVE, SUITE 500, SAN DIEGO, CA, 92130-2332, US

(21) Appl. No.: 09/214,375

(22) PCT Filed: Jul. 18, 1997
(86) PCT No.: PCT/EP97/03961

(30) Foreign Application Priority Data
Jul. 19, 1996 (EP) 96202035

(51) Int. Cl. C12P 23/00

(52) U.S. Cl. 435/67

(57) ABSTRACT

The present invention describes a process to recover highly pure ²-carotene crystals from a crude crystal preparation obtained from a natural source. To remove impurities, the crude crystals are stirred in a solvent in which ²-carotene has a low solubility, whereupon the crystals are filtered off and washed with fresh solvent. Using the process of the invention, a natural crystalline ²-carotene preparation with a very high purity is obtained, a purity comparable to that of synthetic ²-carotene.

### FIELD OF THE INVENTION

The present invention relates to the field of the recovery of carotenoids from a natural source.

### BACKGROUND OF THE INVENTION

Currently, β-carotene crystals with a high purity (96% or higher) are produced by chemical synthesis. When derived from a natural source, β-carotene mostly is in the form of an oily extract (palm oil, algal oil). Although it is also possible to obtain β-carotene crystals from natural sources, such as vegetables (for example carrots) or micro-organisms (for example algae (Dunaliella) or fungi (Blakeslea), the currently available processes to obtain relatively pure crystals from said natural sources have important disadvantages.

Current processes for the purification of β-carotene crystals from a natural source typically comprise an extraction step and additional purification steps.

The extraction is carried out with various extractants: organic solvents, such as ethyl acetate, butyl acetate or hexane, vegetable oils, or supercritical fluids, such as propane, ethylene, CO₂.

To obtain a relatively pure β-carotene preparation, a further purification of the extract is necessary. Several purification processes have been described, among which chromatography, adsorption/desorption processes and crystallization or precipitation.

The β-carotene crystals which are directly crystallized from the extract obtained after solvent extraction of a suitable natural source, e.g. by evaporation of the solvent, typically do not have the desired high purity, e.g. a purity compared to that of synthetic β-carotene. In such cases recrystallizations are required (NL 6411184, U.S. Pat. No. 4,439,629). The main drawback of recrystallization is that a large amount of solvent is required to solubilize the β-carotene. In addition, to recrystallize the β-carotene with a sufficiently high yield, large amounts of antisolvent (precipitating solvent) are necessary as well. Thus, these processes have the disadvantage that large amounts of solvents are required and a considerable loss of β-carotene can easily occur.

### SUMMARY OF THE INVENTION

The present invention discloses a process for the preparation of β-carotene crystals with a purity of at least 90% from a natural source. Said process comprises the following steps:

solvent-extraction of β-carotene from said natural source to produce a β-carotene extract,

crystallization of β-carotene from said extract to produce crude β-carotene crystals,

treatment of said crude β-carotene crystals with a solvent selected from the group of solvents in which β-carotene has a low solubility,

optionally repeating said solvent treatment with the same or a different solvent in which β-carotene has a low solubility,

evaporation of the residual solvent from the crystals.

Optionally, the β-carotene extract obtained after solvent extraction of a natural source is washed with water prior to crystallization of β-carotene.

In a preferred embodiment, the method of the invention provides a crystalline β-carotene preparation from a natural source with a purity of ≧95%.

The method of the invention can be conveniently used to increase the β-carotene content of any impure crystalline β-carotene preparation.

### DETAILED DESCRIPTION OF THE INVENTION

The present invention discloses a process for the recovery of highly pure β-carotene crystals from a natural source with a high yield. According to the invention, highly pure β-carotene crystals are recovered from crude β-carotene crystals using a simple and convenient solvent treatment. The process of the invention comprises a treatment of a crude β-carotene crystal preparation with a solvent in which β-carotene has a low solubility.

The crude β-carotene crystals which are subjected to the process of the invention are obtained by conventional extraction and crystallization technology. Typically, the extraction of β-carotene from a natural source is carried out with organic solvents, vegetable oils or supercritical fluids. The subsequent crystallization or precipitation is performed by methods as cooling, evaporation of the solvent or addition of an antisolvent, followed by filtering off the crystals.

The β-carotene content of the crude crystals mainly depends on the natural source which is used for isolation, whereas the applied crystallization procedure additionally can influence the same. Typically, said β-carotene content varies between 60 and 90%.

Surprisingly, the solvent treatment of the crude crystals according to the invention significantly increases the β-carotene content of the crystals. The crystals obtained after solvent treatment have a β-carotene content of ≧90%, preferably a β-carotene content of ≧92%, more preferably a β-carotene content of ≧94%.

In the process according to the invention, crude β-carotene crystals are treated with a solvent in which β-carotene has a low solubility. The solvent treatment of the invention comprises the steps of stirring the crude β-carotene crystals in said solvent during a time period sufficient to enable dissolution of impurities, filtering off the crystals and washing the crystals several times with fresh solvent. Optionally, the treatment is repeated one or more times. After the final washing step, residual solvent is evaporated.

Solvents which are suitable to use in the treatment according to the invention are solvents in which β-carotene has a low solubility, i.e. a solubility of at most 1 g/l at 25° C. Preferably, the solvent is water or an organic solvent.

When water is used, the pH of the water is not a critical factor for the treatment of the invention, although it is preferred that the pH is below 7. More preferably, the pH of the water is 4-6.

The organic solvent preferably is a lower alcohol or a lower acyl ester thereof, wherein lower is understood to comprise a straight or branched chain of 1 to 5 carbon atoms, or acetone. More preferably, the organic solvent is ethanol or ethylacetate.

The solvent in which β-carotene has a low solubility is applied in a sufficient amount to remove a substantial amount of impurities. Preferably, the solvent to crystal ratio is ≧5 ml solvent per gram crystal, more preferably ≧5-10 ml per gram crystal, most preferably ≧20-40 ml per gram crystal. It is understood that the upper limit of the volume of solvent per gram crystals is not a technical limit but is determined by economical criteria.

The time which is necessary for dissolution of the impurities in the solvent is dependent on the temperature at which the stirring is performed. Typically, the stirring is performed at a temperature below the boiling point of the solvent. Preferably, the stirring is performed at a temperature of 20 to 80° C., more preferably 30 to 60° C., most preferably 50° C.

The solubility of β-carotene in the solvent which is used also may influence the temperature of the stirring process. When using a solvent in which β-carotene has a relatively low solubility, e.g. ethanol, the stirring preferably is performed at an elevated temperature, e.g. 50° C., whereas when using a solvent in which β-carotene has a relatively high solubility, e.g. ethylacetate, the stirring temperature may be choosen in the lower range, e.g. 20 to 25° C.

When using a solvent in which β-carotene has a relatively high solubility, it is a preferred option to keep the stirring temperature at a more elevated level, e.g. 50° C., and to cool the mixture after stirring to a lower temperature, e.g. a temperature of 5 to 20° C., to prevent relatively high losses of β-carotene.

At a temperature of 50° C., a suitable stirring time is for instance about 30 minutes.

The present invention also envisages the option to subsequently treat crude β-carotene crystals with two or more different solvents in which β-carotene has a low solubility.

It is a further option to wash the extract obtained by solvent-extraction of a natural source with water prior to crystallization of β-carotene from said extract. In this embodiment, the amount of water used is not very critical, although it may be relevant to choose said amount such that a good phase separation is obtained.

The present invention provides a simple and convenient method to increase the β-carotene content of β-carotene crystals. The solvent treatment of the invention is advantageously applied to any crystalline β-carotene preparation obtainable from a natural source (plant or a microbial), of which the purity is not as high as desired. In addition, the method of the present invention is applicable to a crude crystal preparation of a natural carotenoid other than β-carotene.

Preferably, the method of the invention is applied to β-carotene crystals obtainable from a microbial source, more preferably from algae or fungi (including yeasts), even more preferably from fungi of the order Mucorales, most preferably from Blakeslea trispora.

In a preferred embodiment of the invention, highly pure, natural β-carotene crystals are obtainable from microbial biomass, e.g. from Blakeslea trispora. Said highly pure, natural, crystalline β-carotene preparation has a purity of ≧95%, preferably ≧96%, more preferably ≧97%, even more preferably ≧98%, most preferably ≧99%. Due to the natural origin of the highly pure, crystalline β-carotene preparation, said preparation may also contain a small amount of other carotenoids, such as γ-carotene. The content of γ-carotene thereby may vary from about 0.5 to about 1.5%.

A preferred process comprises the steps of washing an ethylacetate extract obtained from Blakeslea trispora with water prior to crystallization, crystallization of β-carotene from said washed extract to obtain crude crystals and subsequent treatment of said crude crystals with two different solvents, i.e. firstly with ethylacetate and secondly with ethanol, both solvent treatments occurring at a temperature of 50° C.

Thus, the method of the invention enables the manufacture of a crystalline β-carotene preparation from a natural source which has an extremely high purity, i.e. a purity which is not obtained using conventional technology, i.e. a purity which is ≧95%.

The highly purified β-carotene crystals obtained by the process of the invention are suitable for food, pharmaceutical and cosmetic applications. The natural β-carotene crystals typically are applied as a oily suspension, e.g. a 30% (w/v) suspension of crystals in a vegetable oil, such as soybean oil.

### EXAMPLE 1

### Extraction of β-Carotene from Biomass with Ethyl Acetate

54 g of biomass of Blakeslea trispora, containing 4.3% of β-carotene was mixed with 600 ml of ethyl acetate (Merck, p.a.). The suspension was heated to 50° C. and kept at that temperature during 2 hrs under stirring. Subsequently the suspension was filtrated, yielding an extract with a β-carotene concentration of 1.7 g/l.

The β-carotene content of crystals is analyzed spectrophotometrically (FCC III/Monographs) or with proton-NMR.

### EXAMPLE 2

### Isolation of Highly Pure β-Carotene Crystals from Ethyl Acetate Extract

The extract obtained in Example 1 was concentrated to a β-carotene concentration of 12 g/l by evaporating the solvent at 50° C. under vacuum. The concentrate was cooled to 20° C. and kept at 20° C. during 2 hours under stirring. The crystals were filtered and washed with two cake-volumes of ethyl acetate, yielding 0.7 gram of crude crystal with a purity of 91.9%.

Subsequently the dried crude crystals were stirred for 30 minutes in 35 ml of ethanol at 50° C. After cooling to room temperature, the crystals were filtered off, washed with 2 cake volumes of ethanol and dried under vacuum at room temperature. 0.65 gram of crystals with a purity of 97.0% were obtained.

### EXAMPLE 3

### Isolation of Highly Pure β-Carotene Crystals from Ethyl Acetate Extract

An extract containing 1.7 g/l of β-carotene was prepared as described in Example 1. This extract was concentrated to a β-carotene concentration of 10 g/l by evaporating the solvent at 50° C. under vacuum. The concentrate was cooled to 5° C. and kept at 5° C. during two hours under stirring. The crystals were filtered and washed with two cake-volumes of ethyl acetate, yielding 0.8 gram of crude crystal with a purity of 94.1%.

Subsequently the dried crude crystals were stirred for 30 minutes in 41 ml of ethanol at 50° C. After cooling to room temperature, the crystals were filtered off, washed with 2 cake volumes of ethanol and dried under vacuum at room temperature. 0.78 gram of crystals with a purity of 97.3% were obtained.

### EXAMPLE 4

### Extraction of β-Carotene from Biomass with Hexane

60 g of biomass of Blakeslea trispora, containing 4.3% of β-carotene was mixed with 900 ml of hexane (Merck, p.a.). The suspension was heated to 50° C. and kept at that temperature during 3 hrs under stirring. Subsequently the suspension was filtrated, yielding an extract with a β-carotene concentration of 1.5 g/l.

### EXAMPLE 5

### Isolation of Highly Pure β-Carotene from Hexane Extract

The extract obtained in Example 4 was concentrated to a β-carotene concentration of 10 g/l by evaporating the solvent at 50° C. under vacuum. The concentrate was cooled to 5° C. and kept at 5° C. during 2 hours under stirring. The crystals were filtered and washed with two cake-volumes of hexane, yielding 0.94 gram of crude crystal with a purity of 87.9%.

Subsequently the dried crude crystals were stirred for 30 minutes in 35 ml of ethanol at 50° C. After cooling to room temperature, the crystals were filtered off, washed with 2 cake volumes of ethanol and dried under vacuum at room temperature. 0.85 gram of crystals with a purity of 98.0% were obtained.

### EXAMPLE 6

### Washing of Crude β-Carotene Crystals with Water

Crude crystals with a purity of 85.3% were obtained after extraction of dried Blakeslea trispora biomass with ethyl acetate at 50° C. and a solvent to biomass ratio of 30/1, and subsequent concentration of the extract to a concentration of 15 g/l.

These crude crystals were suspended in demineralized water (20 g/l) and stirred for 30 minutes at 40° C. After filtering off the crystals, the crystals were washed with two cake volumes of ethanol at room temperature and dried under vacuum at room temperature.

Washing with demineralized water at pH 4 yields crystals with a purity of 96.2%, washing with demineralized water at pH 7 or pH 10 yields crystals with a purity of 92%.

### EXAMPLE 7

### Large Scale Isolation of Highly Pure β-Carotene Crystals

210 kg of dried biomass of Blakeslea trispora, containing 4.2% of β-carotene was mixed with 4200 l of ethyl acetate. The suspension was heated to 50° C. and kept at that temperature during 3 hours under stirring. Subsequently the suspension was filtered, yielding an extract with a β-carotene concentration of 1.4 g/l.

The ethyl acetate extract was subsequently mixed with demineralized water of 50° C. in a volumetric ratio of 10/1 (extract/water). After stirring during 15 minutes at 50° C., the layers were separated. The ethyl acetate layer was then concentrated to a β-carotene content of 6 g/l by evaporation.

The concentrate was cooled to 5° C., causing crystallization of β-carotene. After two hours at 5° C. under stirring, the crystals were filtered and washed with two cake volumes of ethyl acetate, yielding 6.4 kg of wet crystals (dry matter 60.2%). The wet crystals were stirred in 75 l of ethanol at 50° C. during 30 minutes. The suspension was then allowed to cool to 20° C., the crystals were filtered and subsequently washed with two cake volumes of ethanol. The crystals were finally dried under vacuum at room temperature, yielding 3.6 kg of crystals with a purity of 97.2%.

### EXAMPLE 8

### Large Scale Isolation of Highly Pure β-Carotene Crystals

400 kg of dried biomass of Blakeslea trispora, containing 4.2% of β-carotene was mixed with 12 m³ of ethyl acetate. The suspension was heated to 50° C. and kept at that temperature during 8 hours under stirring. Subsequently the suspension was filtered, yielding an extract with a β-carotene concentration of 1.7 g/l.

The ethyl acetate extract was subsequently mixed with demineralized water of 50° C. in a volumetric ratio of 5/1. After stirring during 15 minutes at 50° C., the layers were separated. The ethyl acetate layer was then concentrated to a β-carotene content of 4 g/l by evaporation.

The concentrate was cooled to 5° C., causing crystallization of β-carotene. After two hours at 5° C. under stirring, the crystals were filtered and washed with two cake volumes of ethyl acetate, yielding 24 kg of wet crystals (dry matter 55%). The wet crystals were subsequently stirred in 450 l of ethyl acetate at 50° C. during 30 minutes. The suspension was then allowed to cool to 5° C., the crystals were filtered and subsequently washed with two cake volumes of ethyl acetate and finally one cake volume of ethanol. 23 kg of wet crystals were obtained.

Next the crystals were stirred in 450 l of ethanol (95%) at 50° C. during 30 minutes. After cooling of the suspension to 20° C., the crystals were filtered and washed with two cake volumes of ethanol, yielding 23 kg of wet crystals. The crystals were finally dried under vacuum at room temperature, yielding 13.8 kg of crystals with a purity of 100.4% (spectrophotometric method of FCC III).

1. A process for the preparation of β-carotene crystals with a purity of ≧90% from a natural source, comprising the steps of:
solvent-extraction of β-carotene from said source to produce a β-carotene extract,
crystallization of β-carotene from said extract to produce crude β-carotene crystals,
treatment of said crude β-carotene crystals with a solvent selected from the group of solvents in which β-carotene has a low solubility,
optionally repeating the previous solvent treatment with the same or a different solvent in which β-carotene has a low solubility,
evaporation of the residual solvent from the crystals.

2. The process of claim 1, wherein a first solvent treatment of crude β-carotene crystals is followed by a second treatment with a solvent different from the first solvent.

3. The process of claim 1 or 2, wherein the treatment of said crude β-carotene crystals comprises the steps of:
stirring said crude β-carotene crystals in said solvent,
filtering off the crystals,
washing the crystals with fresh solvent.

4. The process of any one of the claims 1-3, wherein the solvent in which β-carotene has a low solubility is water.

5. The process of any one of the claims 1-3, wherein the solvent in which β-carotene has a low solubility is an organic solvent.

6. The process of claim 5, wherein said organic solvent is selected from the group of lower alcohols and acetone.

7. The process of claim 6, wherein said organic solvent is selected from the group of ethanol and ethyl acetate.

8. The process of any one of the claims 1-7, wherein the β-carotene extract is washed with water prior to crystallization.

9. The process of any one of the previous claims, wherein the natural source is a microbial source.

10. The process of claim 9, wherein the microbial source is a fungus or an alga.

11. The process of claim 10, wherein the fungus is from the order Mucorales.

12. The process of claim 11, wherein the fungus is Blakeslea trispora.

13. A crystalline β-carotene preparation obtainable from a natural source with a purity of ≧95%, preferably ≧96%, more preferably ≧97%, even more preferably ≧98%, most preferably ≧99%.

14. The β-carotene preparation of claim 13, wherein the natural source is a microbial source.

15. The β-carotene preparation of claim 14, wherein the microbial source is a fungus or an alga.

16. The β-carotene preparation of claim 15, wherein the fungus is from the order Mucorales.

17. The β-carotene preparation of claim 16, wherein the fungus is Blakeslea trispora.
